(19) United States
(12) Patent Application Publication
(10) Pub. No.: US 2024/0307529 A1
(43) Pub. Date: Sep. 19, 2024

(54) COMPOSITION FOR IMMUNE TREATMENT OF ALLERGIC DISEASES COMPRISING HOUSE DUST MITE ANTIGENS AND HYALURONIC ACID HYDROGELS AS ACTIVE INGREDIENTS

(71) Applicant: THE CATHOLIC UNIVERSITY OF KOREA INDUSTRY-ACADEMIC COOPERATION FOUNDATION, Seoul (KR)

(72) Inventors: Yoon Hong CHUN, Seoul (KR); Hyun Jong LEE, Gyeonggi-do (KR); Kyung Sun NA, Seoul (KR); Ji An KIM, Seoul (KR)

(21) Appl. No.: 18/370,598

(22) Filed: Sep. 20, 2023

(30) Foreign Application Priority Data
Mar. 17, 2023 (KR) 10-2023-0035411

(51) Int. Cl. A61K 39/35; A61K 39/00; A61K 41/00; A61P 37/08

(52) U.S. Cl. CPC A61K 39/35; A61K 39/0003; A61K 41/0042; A61P 37/08; A61K 2039/6087; A61K 2039/6093

(57) ABSTRACT

The present invention relates to a composition for immunotherapy of allergic diseases, comprising house dust mite antigens and hyaluronic acid hydrogels as active ingredients. By using hyaluronic acid as a material, and forming a crosslinking with the house dust mite antigens via a photoinitiator and visible light, it can overcome the disadvantage of easy degradation and simultaneously exploit biocompatible features. Moreover, it can provide a large amount of antigens to immune cells with a single administration, and despite this, it does not induce foreign body reactions, drug reactions, or immune responses, while generating excellent immune tolerance effects. Its safety is outstanding and it does not require repeated injections over a long period. Thus, it is expected to be useful as a preventive or therapeutic agent for allergic diseases induced by house dust mites.

### CROSS-REFERENCE TO RELATED APPLICATION

The application claims priority from Korean Patent Application No. 10-2023-0035411 filed on Mar. 17, 2023. The entire disclosure of the application identified in this paragraph are incorporated herein by reference.

### FIELD

The present invention relates to a composition for immunotherapy of allergic diseases, comprising house dust mite antigens and hyaluronic acid hydrogels as active ingredients.

### BACKGROUND

House dust mites have been confirmed to cause sensitization to antigens in 79% of allergic rhinitis and asthma patients, and they are known to be the largest cause of respiratory diseases due to allergies. Particularly, allergic rhinitis induces chronic cough, nasal congestion, and rhinorrhea, as well as chronic fatigue, decreased physical ability, and concentration, which interfere with daily tasks and learning. If left untreated for a long time, it can lead to other allergic diseases such as allergic asthma and allergic conjunctivitis. These allergic diseases require a long period of rest and drug administration for symptom improvement and recovery, thus causing significant socioeconomic costs.

Current treatments for allergic rhinitis comprise nasal steroids, oral steroids, and antihistamines used to alleviate inflammation in the nasal mucosa. However, long-term use of steroids can lead to decreased immunity, endocrine disruption, stunted growth, and weight gain. Antihistamines only provide temporary relief of symptoms and are not a fundamental cure, and side effects such as drowsiness, decreased concentration, and constipation have been reported.

Allergen-specific immunotherapy is the only known curative treatment for allergic diseases, but conventional subcutaneous injections and sublingual methods require a long maintenance period of 3 to 5 years before effects appear, and many patients give up treatment before sufficient effects are seen due to low patient compliance. In classical immunotherapy, it takes a long time to increase the dose to reach the maintenance dose for safe administration, and the maintenance treatment process also requires inconvenient repetition of sublingual administration once or twice a week or subcutaneous injections at biweekly intervals, leading to low treatment success rates. Ultra-rush immunotherapy can be implemented to solve these problems, but many patients experience dangerous anaphylaxis reactions during treatment, so it is only implemented in special cases in actual clinical practice.

Meanwhile, hydrogels are typically composed of natural polymers such as hyaluronic acid (HA), collagen, gelatin, and cellulose, forming a three-dimensional network structure of hydrophilic polymer chains, and they are completely degraded and absorbed in the body. Hydrogels also have unique mechanical and structural characteristics similar to the tissues and extracellular matrix (ECM) of the skin, so hydrogels do not cause foreign body reactions or inflammatory reactions. Due to these characteristics of hydrogels, they are already used as biocompatible materials in various fields such as tissue engineering, imaging, treatment, and medical devices.

If allergens and hydrogels can be combined and delivered into the body in a safe manner, a large amount of antigen can be provided to immune cells without inducing abnormal immune responses over a long period with a single administration, thus inducing immune tolerance to allergens. Also, it can increase treatment compliance by reducing the frequency of administration and providing a safe method. However, no such method for immunotherapy has been reported so far. The present invention is an effective and safe new method of immunotherapy, which is a hyaluronic acid hydrogel therapeutic agent combined with a house dust mite allergen that forms a photo-crosslinking in the human body, and can overcome the drawbacks of traditional immunotherapies.

### PRIOR ART DOCUMENTS

### Patent Documents

(Patent Document 1) KR 10-2016-0096206

### SUMMARY

### Technical Problem

One object of the present invention is to provide a composition for immunotherapy of allergic diseases, which comprises a crosslinked product of house dust mite antigens and hyaluronic acid hydrogels as active ingredients.

Another object of the present invention is to provide a kit for the immunotherapy of allergic diseases, which comprises a crosslinked product of house dust mite antigens and hyaluronic acid hydrogels, and instructions.

However, the technical tasks that the present invention intends to achieve are not limited to the tasks mentioned above, and other tasks not mentioned can be clearly understood by those skilled in the art from the description below.

### Technical Solution

The present invention provides a composition for immunotherapy of allergic diseases, which comprises a crosslinked product of house dust mite antigens and hyaluronic acid hydrogels as active ingredients.

The present invention provides a kit for immunotherapy of allergic diseases, which comprises a crosslinked product of house dust mite antigens and hyaluronic acid hydrogels, and instructions.

In one embodiment of the present invention, the antigen may be a crude extract, but is not limited thereto.

In one embodiment of the present invention, the composition may further comprise a photoinitiator, but is not limited thereto.

In one embodiment of the present invention, the photoinitiator may be riboflavin, but is not limited thereto.

In one embodiment of the present invention, the crosslinking of the crosslinked product may be by visible light, but is not limited thereto.

In one embodiment of the present invention, the house dust mite may be one or more selected from the group consisting of Dermatophagoides pteronyssinus, Dermatophagoides farinae, Dermatophagoides microceras, and Euroglyphus maynei, but is not limited thereto.

In one embodiment of the present invention, the composition may further comprise an adjuvant, but is not limited thereto.

In one embodiment of the present invention, the allergic disease may be induced by house dust mites, but is not limited thereto.

In one embodiment of the present invention, the allergic disease may be one or more selected from the group consisting of rhinitis, asthma, conjunctivitis, urticaria, contact dermatitis, and atopic dermatitis, but is not limited thereto.

Moreover, the present invention provides an immunotherapy use for allergic diseases of a crosslinked product of house dust mite antigens and hyaluronic acid hydrogels or a composition comprising the same an active ingredient.

In addition, the present invention provides a method for immunotherapy of allergic diseases, which comprises the step of administering a crosslinked product of house dust mite antigens and hyaluronic acid hydrogels or a composition comprising the same as an active ingredient to an individual in need thereof.

Furthermore, the present invention provides a use for manufacturing a preparation for immunotherapy of allergic diseases of a crosslinked product of house dust mite antigens and hyaluronic acid hydrogels or a composition comprising the same as an active ingredient.

### Advantageous Effects

A composition for preventing or treating an allergic disease, comprising house dust mite antigens and hyaluronic acid hydrogels as active ingredients can form a crosslink with house dust mite antigens using hyaluronic acid as a material and with a photoinitiator and visible light, making administration possible through an injection and overcoming the drawback of easily decomposing in a short time. In addition, it can provide a large amount of antigens to immune cells with a single administration, but it does not induce foreign body reaction and abnormal immune reaction, so it has high safety, excellent immune tolerance effect, and does not require frequent injections, thus, it is expected to be usefully utilized for preventing or treating of allergic diseases induced by house dust mites.

### BRIEF DESCRIPTION OF THE DRAWINGS

FIG. 1 is a schematic diagram showing the mechanism of action following subcutaneous administration of the HA hydrogel-HDM therapeutic agent.

FIG. 2A is a photograph taken through a scanning electron microscope of the cross section of the injectable HA hydrogel.

FIG. 2B is a graph showing the results of degradation analysis to evaluate the properties of the injectable HA hydrogel.

FIGS. 2C and 2D are photos and graphs observing the size change of the gel over time after crosslinking with a blue light following subcutaneous administration of the injectable photo-crosslinking HA hydrogel to mice.

FIG. 3A is a graph showing the results of the degradation speed analysis of the SH-HA+MA-HA hydrogel depending on the presence of HDM.

FIG. 3B is a graph showing the results of antigen release amount analysis due to the degradation of the HA hydrogel-HDM immunotherapeutic agent.

FIGS. 4A and 4B are schematic diagrams showing the allergy rhinitis animal experiment protocol to verify the immune therapeutic effect of the HA hydrogel-HDM immunotherapeutic agent (each of negative control, positive control, conventional SCIT treatment group, HDM gel treatment group).

FIG. 4C is a graph showing the allergy symptom relief effect according to the HA hydrogel-HDM immunotherapeutic agent treatment.

FIG. 5 is a graph showing the immune globulin increase and decrease effect according to the HA hydrogel-HDM immunotherapeutic agent treatment.

FIG. 6A are histopathological feature of nasal mucosa with H&E staining, toluidine blue staining, and PAS staining according to the HA hydrogel-HDM immunotherapeutic agent treatment.

FIG. 6B graphs conducted to compare the infiltration of allergic inflammation cells in nasopharyngeal lavage analysis and the nasal mucosa.

FIGS. 7A, 7B and 7C are graphs showing the cytokine increase and decrease effect according to the HA hydrogel-HDM immunotherapeutic agent treatment.

### DETAILED DESCRIPTION

The composition of the present invention utilizes hydrogels as a delivery material for house dust mite antigens. The immunotherapeutic agent using injectable HA hydrogels employed in the present invention allows easy subcutaneous injection of a liquid hydrogel containing a large amount of allergens. Upon exposure to visible light or blue light, this liquid hydrogel transforms into a viscous hydrogel, thereby slowing its degradation. This ensures a consistent degradation of an allergen load that can induce immune tolerance without triggering anaphylaxis. Furthermore, the consistent rate of degradation of the allergen-loaded hydrogel resolves the inconvenience of frequently administering allergens through subcutaneous injection. Before photo-crosslinking occurs, the hydrogel remains in a liquid state, allowing the use of thin injection needles, which feature the advantage of not causing pain during the injection process. These features are expected to solve all the limitations of subcutaneously injected immunotherapeutic agents used so far. The manufacturing costs and safety of administration are expected to be superior to other immunotherapeutic agents, indicating a high likelihood of commercialization.

The present invention provides an improved immunotherapeutic composition for treating allergic diseases, which comprises the crosslinked product of house dust mite antigens and hyaluronic acid hydrogels as active ingredients.

In the present invention, the term “house dust mite antigens” may comprise any components derived from house dust mites that can cause allergic reactions without limitation, but is not limited thereto. For example, in one embodiment of the present invention, the antigens may be crude extracts, but are not limited thereto, and it comprises all house dust mite antigens comprising crude extracts.

In the present invention, the term “crude extract” refers to the product initially obtained in the extraction process. House dust mite crude extract may mean antigens in an unrefined state, initially or ultimately, in the manufacture of house dust mite antigens. Generally, in the present industry, crude extracts not only contain major allergens but also a number of other substances, hence the protein amount is not constant, allergenicity is not constant, and they can cause unpredictable allergies or symptoms, which are significant disadvantages, and are therefore not typically used for immunotherapy of allergic diseases. However, when using the composition according to the present invention, it can show the same or even superior immunotherapeutic effects for allergic diseases as using the ultimately refined house dust mite antigens.

In one embodiment of the present invention, the composition may further comprise a photoinitiator, but is not limited thereto. In the present invention, a photoinitiator is a substance that can form radicals by irradiation with light to cause photopolymerization, and any photoinitiator that can be used for conventional photopolymerization can be used. In one embodiment of the present invention, the photoinitiator may be riboflavin, but is not limited thereto.

In one embodiment of the present invention, the crosslinking of the crosslinked product may be by visible light, but is not limited thereto.

In the present invention, visible light refers to the region of the electromagnetic spectrum visible to the human eye, which is generally known to detect a range from 400 to 700 nm. The composition of the present invention forms crosslinks through a photoinitiator, and compared to ultraviolet and infrared light, the composition can be easily manufactured by conveniently irradiating visible light.

In one embodiment of the present invention, the house dust mite may be one or more selected from the group consisting of Dermatophagoides pteronyssinus, Dermatophagoides farinae, Dermatophagoides microceras, and Euroglyphus maynei, but is not limited thereto.

In one embodiment of the present invention, the composition may further comprise an adjuvant, but is not limited thereto.

In the present invention, “adjuvant” comprises a substance that enhances the immune response to an antigen and is used in purified antigen vaccines exhibiting a low immune response, enabling induction of a high immune response even with low concentrations of antigens, and it represents the broadest meaning, encompassing the commonly used meaning in the immunotherapy industry.

In one embodiment of the present invention, the allergic disease may be induced by house dust mites, but is not limited thereto.

In this specification, the term “allergy” generally refers to symptoms where the immune system overreacts to antigens that are not harmful to the body. When an allergy is triggered by an antigen, symptoms such as urticaria, itching, rhinorrhea, and cough occur, and in severe cases, it can cause anaphylaxis reactions, causing respiratory difficulty, acute hypotension, and shock. The causative substance of allergies, “allergen”, varies from pollen, antibiotics, food, etc., and house dust mites are also known to cause serious allergies by stimulating the respiratory tract and mucous membranes of the human body with their bodies and excrement, promoting the production of histamines and others, but is not limited thereto.

In the present invention, “house dust mite” is a mite species that breeds in dust in residential areas, generally measuring only 0.2 to 0.3 mm in size, with an opaque body, making it almost impossible to observe with the naked eye. Representative species comprise Dermatophagoides pteronyssinus, Dermatophagoides farinae, Dermatophagoides microceras, and Euroglyphus maynei, but this is not limited thereto.

In the present invention, “allergic diseases” refer to diseases caused by allergies, which may be allergic diseases in particular caused by house dust mites. In one embodiment of the present invention, the allergic disease may be one or more selected from a group consisting of rhinitis, asthma, conjunctivitis, urticaria, contact dermatitis, and atopic dermatitis, but is not limited thereto.

In the present invention, “immunological tolerance” refers to a state where the organism does not respond to antigens that trigger an immune response. In other words, immunological tolerance can prevent or alleviate allergic reactions by suppressing the immune system's overreaction to allergens. Therefore, an immunotherapeutic agent intended for preventing and/or treating allergic diseases aims to induce immunological tolerance to a given antigen by exposing the immune system to an appropriate level of the antigen. The pharmaceutical composition according to the present invention, which comprises hydrogel components combined with a house dust mite crude extract (or antigen), can accustom the immune system to house dust mite antigens and induce immunological tolerance, even with a single administration of a small quantity of house dust mite antigens. Consequently, it demonstrates excellent immunotherapeutic effects for house dust mite allergic diseases, but is not limited thereto.

The pharmaceutical composition according to the present invention may further comprise a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be comprised in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, methylcellulose, carboxymethylcellulose, sodium tragacanth, carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may comprise: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, S-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration comprise tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration comprise suspensions, liquids for internal use, emulsions, syrups, and the like, and these liquid preparations may comprise, in addition to simple commonly used diluents, such as water and liquid paraffin, various types of excipients, for example, a wetting agent, a sweetener, a fragrance, a preservative, and the like. Preparations for parenteral administration comprise an aqueous sterile solution, a non-aqueous solvent, a suspension, an emulsion, a freeze-dried preparation, and a suppository. Non-limiting examples of the non-aqueous solvent and the suspension comprise propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors comprising types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases. However, since the effective amount may be increased or decreased depending on the administration route, the severity of disease, gender, body weight, age, and the like, the dosage is not intended to limit the scope of the present invention in any way.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method. The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “improvement” as used herein means all actions that reduce the degree of parameters related to a target disease, e.g., symptoms via administration of the composition according to the present invention.

The present invention provides a kit for immunotherapy of allergic diseases, comprising a crosslink of house dust mite antigens and hyaluronic acid hydrogels, and instructions.

The “kit” of the present invention can comprise other components, devices, substances, etc., typically necessary for the method of immunotherapy for allergic diseases caused by house dust mites, in addition to the composition mentioned. Furthermore, all components comprised in the kit can be used without restriction to the number of uses, there are no restrictions before and after using each material, and the application of each material can proceed simultaneously or at different times.

The kit of the present invention can comprise a container, in addition to the composition and the instructions. The container can serve to package the composition and also to store and fix it. The material of the container can, for example, take the form of a bottle, tub, sachet, envelope, tube, ampoule, etc., and these can be formed partially or entirely from plastic, glass, paper, foil, wax, and the like. The container may be equipped with a fully or partially removable closure, which may be initially part of the container or may be attached to the container by mechanical, adhesive, or other means, and may also be equipped with a stopper that allows access to the contents by a syringe needle. The kit can comprise an external package, which may contain instructions for the use of the components, but is not limited thereto.

Hereinafter, preferred examples are provided to facilitate understanding of the present invention. However, the following examples are provided to facilitate understanding of the present invention, and the content of the present invention is not limited by following examples.

### EXAMPLES

### Example 1. Manufacture of Injectable HA Hydrogel-HDM Immunotherapeutic Agent

### Example 1-1. Manufacture of Injectable HA Hydrogel

SH-HA+MA-HA gel was prepared by dissolving each of thiolated and methacrylated hyaluronic acids in 1 ml of PBS at 10 mg/mL, then adjusting the pH to 7 using sodium hydroxide. The pH-adjusted solution was placed into a microtube so that the volume ratio of methacrylate to thiol was 1:1, and riboflavin was appropriately added to make up 0.01% of the total volume, thereby producing a hydrogel solution.

4-arm PEG-SH+MA-HA gel was prepared by dissolving 4-arm PEG-SH and methacrylated hyaluronic acid in 1 ml of PBS at 10 mg/mL, then adjusting the pH to 7 using sodium hydroxide. The pH-adjusted solution was placed into a microtube so that the volume ratio of 4-arm PEG-SH to HA-MA was 1:1, 2:1, or 4:1, and riboflavin was appropriately added to make up 0.01% of the total volume, thereby producing a hydrogel solution.

Each prepared solution was exposed to blue light for 10 to 20 seconds to initially form the hydrogel. Since blue light only takes about 10 seconds to form a gel, the appropriate time was selected depending on the situation.

### Example 1-2. Manufacture of HA Hydrogel-HDM Immunotherapeutic Agent

In Example 1-1, HDM was additionally mixed with the prepared SH-HA+MA-HA and 4-arm PEG-SH+MA-HA hydrogel precursor solutions to produce HDM-comprised HA hydrogel precursor solutions. HDM antigen (Dermatophagoides pteronyssinus) was sourced from GREER laboratories, Lenoir, NC, USA.

For gel treatment group, gel-1 and gel-2, HDM antigen 750 μg was mixed with 100 μL of liquid HA hydrogel precursor to produce a dosage for one-time subcutaneous injection, which was used in animal experiments. After subcutaneous injection, it was exposed to blue light for 20 seconds to transform it into a solid gel state.

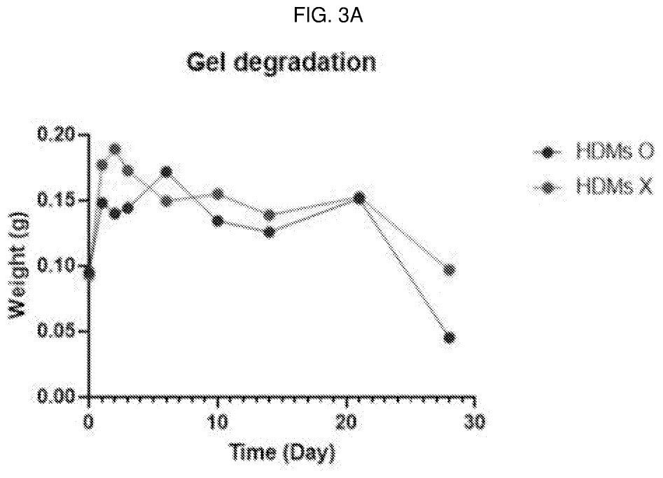

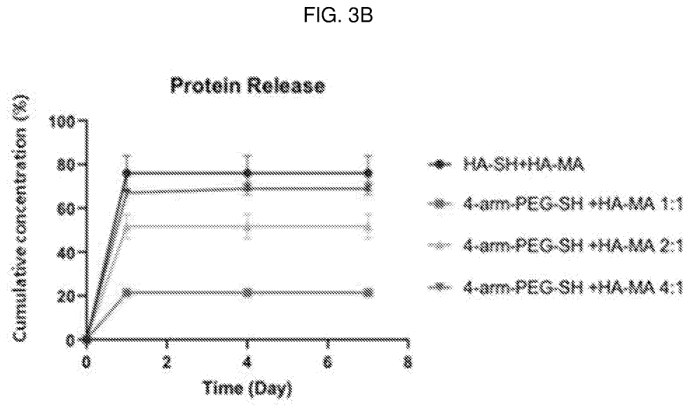

Since the gel can be formed by exposing it to blue light for about 10 seconds, the rate of degradation was compared according to the amount of hydrogel administered and the exposure time of the blue light as described in Example 3-1, and finally 100 μl, 20 seconds were applied to the experiment (FIG. 3A and FIG. 3B).

### Example 2. Reforming and Property Evaluation of Injectable HA Hydrogels

The reforming and physical properties of the injectable HA hydrogels manufactured according to Example 1 were analyzed. Injectable HA hydrogels utilize a reaction known as photo-click chemistry, which utilizes a thio-ene reaction. Depending on the ratio of thiol and acrylate functional groups, the degradation and drug delivery speed can be adjusted. Therefore, hydrogels with four different functional groups were produced, and the structure and degradation rate were analyzed through electron microscope images to verify whether the immunotherapeutic agent of the present invention can maintain a suitable form for drug delivery while also undergoing appropriate degradation.

### Example 2-1. Analysis of Reforming of Injectable HA Hydrogels

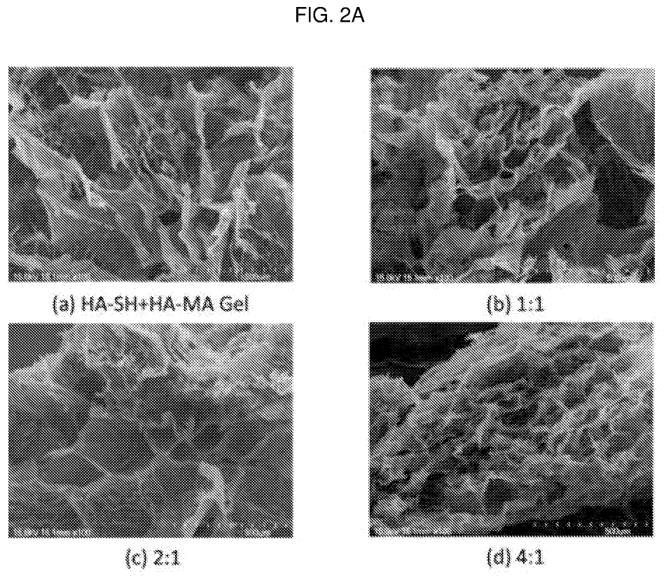

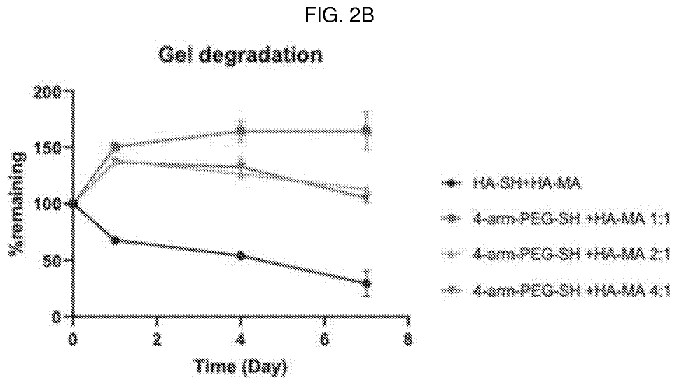

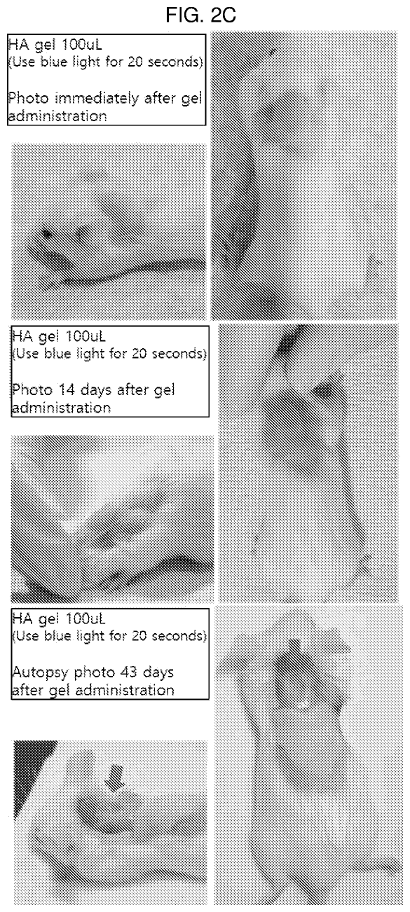

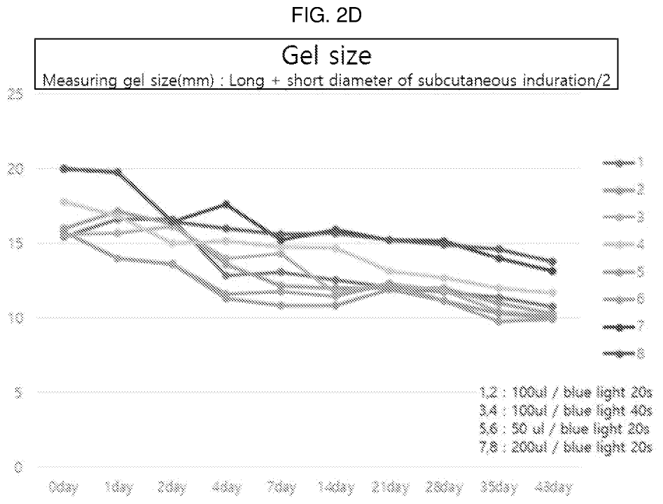

To analyze the reforming of the injectable HA hydrogels, the cross-section of the hydrogel was examined through a scanning electron microscope (SEM). Specifically, the hydrogel was frozen with liquid nitrogen immediately after production, the section was cut, freeze-dried for 24 hours, and then photographed after being coated with Pt for 100 seconds. The results are shown in FIG. 2A.

### Example 2-2. Property Evaluation of Injectable HA Hydrogels

To evaluate the physical properties of the injectable HA hydrogels, the extent of degradation of the hydrogel was analyzed. The degree of degradation was determined by measuring the weight. Specifically, to measure the degree of degradation, the hydrogels were made and stored in water, and at certain measurement points, all the water was removed and weighed, after which the hydrogels were refilled with water and stored.

As a result, 4-arm PEG-SH+HA-MA 1:1 was found to absorb water and expand, showing an increase from the initial weight, and no decrease in weight was observed for up to 7 days, confirming that degradation hardly proceeded. Also, 4-arm PEG-SH+HA-MA 2:1 and 4:1 were found to show almost similar patterns of weight change. Specifically, the weight increased on the first day by absorbing water, but over time, the weight gradually decreased, indicating that the degradation of the hydrogel also gradually occurred. SH-HA+MA-HA showed a decreased weight even on the first day, and a continuous decrease in weight was confirmed for 7 days, confirming the fastest degradation pattern (FIG. 2B).

### Example 2-3. Confirmation of In Vivo Reduction Degree of Injectable HA Hydrogel

Based on Examples 2-1 and 2-2, the SH-HA+MA-HA gel, expected to deliver the drug the fastest, and 4-arm PEG-SH+HA-MA 1:1, expected to deliver the drug the slowest, were selected to analyze the in vivo reduction degree of the injectable HA hydrogel of the present invention through animal experiments.

A preliminary experiment was conducted by injecting the gel into mice to check the rate of absorption into the surrounding tissue and the presence of inflammation after subcutaneous injection of the injectable HA hydrogel. Specifically, after injecting 50 or 100 μL of HA hydrogel into BALB/c mice, it was checked whether a photoinitiation reaction occurs when visible light was shined for 20 seconds and 40 seconds, and the degree of reduction of the hydrogel over time after injection was checked, and finally, an autopsy was conducted 6 weeks (43 days elapsed) later to visually check the condition of the surrounding tissue.

As a result, it was confirmed that when visible light was shined on the surface of the skin after injecting the HA hydrogel into the subcutaneous layer of the mouse, a photoinitiator reaction occurred, forming a suitable solid. After 43 days, a necropsy of the mouse revealed that when 100 μL of the HA hydrogel and 20 seconds of blue light were used, the size of the hydrogel remained about 50% of the initial injection amount (FIG. 2C and FIG. 2D). Furthermore, the HA hydrogel did not adhere to surrounding tissues, and the surrounding subcutaneous tissues did not exhibit inflammation or a foreign body reaction when compared with parts not in contact with the gel. This suggests that the injectable HA hydrogel is expected to be safely degraded and absorbed in the body.

### Example 3. Verification of the Degradation Effect of HA Hydrogel-HDM Immunotherapeutic Agent

In order to confirm the degradation effect of the HA hydrogel-HDM immunotherapeutic agent according to the present invention, the degradation rate and degradation activity were each analyzed.

### Example 3-1. Verification of the Degradation Rate of HA Hydrogel-HDM Immunotherapeutic Agent

An in vitro experiment was conducted to analyze whether there was a difference in the degradation rate when HDM was attached and not attached using the SH-HA+MA-HA gel. Specifically, the gel was immersed in PBS at a temperature of 37° C., the same as body temperature, and removed at a fixed time to measure the weight of the gel.

As a result, a similar degradation rate was observed up to 28 days of observation regardless of the presence or absence of the HDM, which is a crude extract, and it was confirmed that the gel maintained the same viscosity and physical properties when touched on the 28th day regardless of the presence or absence of HDM (FIG. 3A).

### Example 3-2. Verification of Antigen Release of HA Hydrogel-HDM Immunotherapeutic Agent

The release of protein antigens due to the degradation of the HA hydrogel-HDM immunotherapeutic agent according to the present invention was analyzed. Specifically, HA hydrogel-HDM immunotherapeutic agents made from SH-HA+MA-HA 1:1 gel, 4-arm PEG-SH+MA-HA 1:1 gel, 4-arm PEG-SH+MA-HA 2:1 gel, and 4-arm PEG-SH+MA-HA 4:1 gel were prepared (SH-HA is Thiolated Hyaluronic Acid, MA-HA is Methacrylated Hyaluronic Acid, 4-arm PEG-SH is 4-Arm PEG-Thiol). The difference in HDM release from the immunotherapeutic agent gel manufactured according to each ratio was compared. The protein was quantified by the BCA assay, and the solution was taken at regular dates to quantify and analyze the protein contained in the solution.

As a result, each immunotherapeutic agent released a certain amount of HDM primarily according to the composition of the gel up to the initial 24 hours, and from the second day onwards, it exhibited a trend of being maintained constantly (FIG. 3B). Specifically, the degree of gel degradation according to the functional group appeared in the order of SH-HA+MA-HA, 4-arm PEG-SH+MA-HA 4:1 gel, 4-arm PEG-SH+MA-HA 2:1 gel, 4-arm PEG-SH+MA-HA 1:1 gel. Such a result suggests that the release speed and degradation speed of HDM antigen are similar in that they match the release speed of HDM antigen due to gel degradation. In particular, for SH-HA+MA-HA 1:1 gel with a fast degradation rate, 80% of HDM was released in the initial two days, while for 4-arm PEG-SH+MA-HA 1:1 gel with a slow degradation rate, only 20% of HDM was released over a week, and it was confirmed that it does not release HDM and maintains it until additional degradation occurs.

According to the results, the existing injectable immunotherapeutic agents induce immune tolerance to allergens by periodically administering antigens, but when a large amount of house dust mite antigens are combined with a hydrogel composed of a three-dimensional porous structure and administered, the contact between the antigens inside the gel and the immune cells is limited, thus preventing the occurrence of abnormal immune reactions such as anaphylaxis. Over time, as the natural degradation of the hydrogel proceeds, the stepwise release of antigens occurs, and it has been confirmed that a periodic immune response can be induced for a long period of time with a single injection.

### Example 4. Verification of Allergy Symptom Relief Effect of HA Hydrogel-HDM Immunotherapeutic Agent

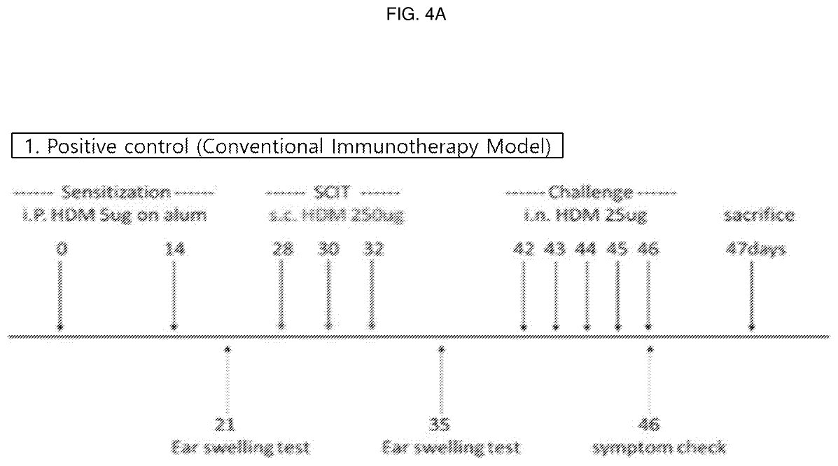

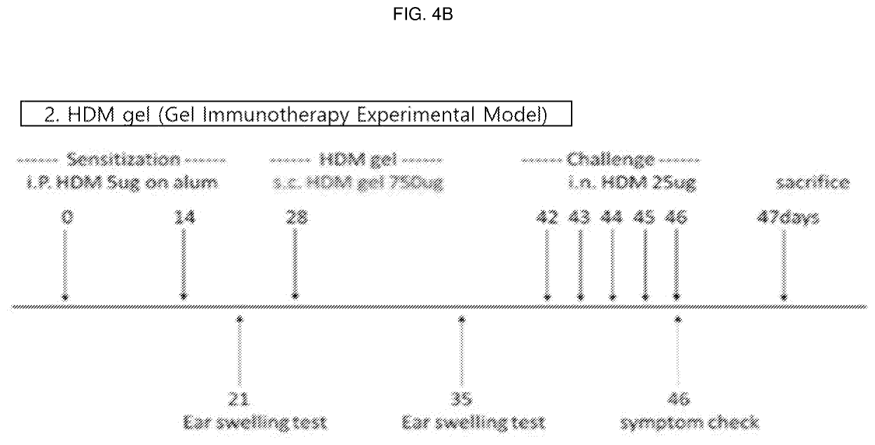

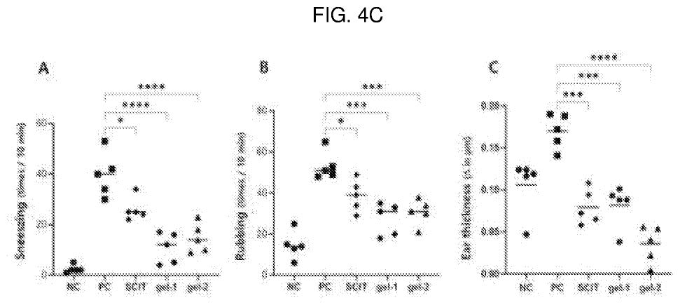

In order to verify the allergy symptom relief effect of the HA hydrogel-HDM immunotherapeutic agent of the present invention, an allergic rhinitis induction model was produced. Specifically, the conventional subcutaneous immunotheraphy (SCIT) model was used for effect comparison according to FIG. 4A. In addition, the most rapidly degrading SH-HA+MA-HA (gel-1 group) and the slowest degrading 4-arm PEG-SH+MA-HA 1:1 (gel-2 group) in Example 3 were applied to the allergic rhinitis induction model according to FIG. 4B to compare the symptom relief effects (Table 1). All groups used an animal rectal thermometer to measure rectal temperature before and after administration of HDM crude extract or HDM mixed with HA hydrogel, and confirmed that there was no significant change in body temperature before and after administration, confirming that an anaphylaxis reaction did not occur. Subsequently, nasal rubbing (A) and sneezing count (B) were measured for 10 minutes after exposure to house dust mites, and the reduction in edema (C) was confirmed by injecting HDM into the ears of mice after immunotherapy.

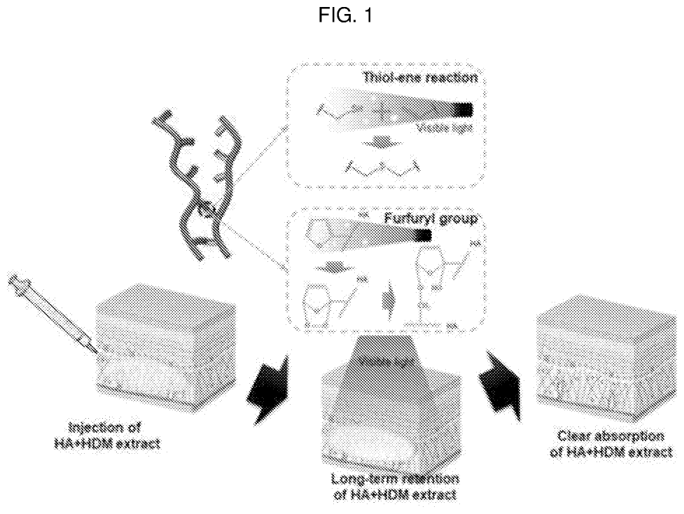

TABLE 1

| Classification | Content |
|---|---|
| Negative Control | Negative control (HDM sensitization) |
| Group | |
| Positive Control | Positive control (HDM sensitization + HDM |
| Group | challenge) |
| SCIT Group | Positive control + conventional subcutaneous |
| | immunotherapy with HDM according to FIG. 1 |
| Gel 1 Group | Positive control + SH-HA + MA-HA gel with HDM |
| | according to FIG. 2 |
| Gel 2 Group | Positive control + 4-arm PEG-SH + MA-HA 1:1 gel |
| | with HDM according to FIG. 2 |

As a result, it was found that in the gel-1 group and gel-2 group, all nasal rubbing (A), sneezing count (B), and edema occurrence in the skin after HDM stimulation (C) significantly decreased compared to the positive control group, and it was confirmed that this is remarkably superior to the conventional treatment method SCIT group (FIG. 4C). (NC, negative control; PC, positive control; SCIT, conventional subcutaneous immunotherapy with HDM; Gel-1, SH-HA+MA-HA gel with HDM; Gel-2, 4-arm PEG-SH+MA-HA 1:1 gel with HDM. * P<0.05; ** P<0.01; *** P<0.001;**** P<0.0001).

### Example 5. Verification of the Immunoglobulin Modulation Effect of the HA Hydrogel-HDM Immunotherapeutic Agent

The impact of the HA hydrogel-HDM immunotherapeutic agent, manufactured in accordance with Example 1, on immunoglobulins was investigated using the same experimental model and groups as in Example 4. Following immunotherapy, the changes in mouse total IgE, HDM specific IgE, HDM specific IgG1, and HDM specific IgG2 levels in the serum were analyzed.

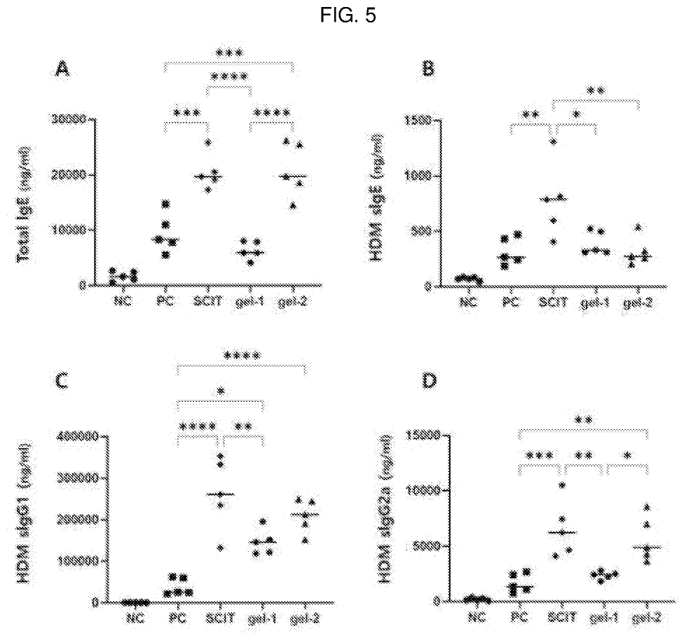

The results are shown in FIG. 5. Specifically, in the case of total IgE and specific IgE, which are closely related to the onset of allergic symptoms, it is known that they inevitably increase in conventional immunotherapy that requires direct injection of the allergen, separate from the therapeutic effect. In the present invention, the gel-1 group exhibited significantly lower total IgE and specific IgE levels compared to the SCIT group, and the gel-2 group also demonstrated significantly lower specific IgE levels, indicating that unavoidable allergic reactions did not occur (FIGS. 5A and B).

Additionally, in the case of IgG1, an immunoglobulin that increases due to immunotherapy and determines the protective effect against allergic symptoms, both the gel-1 and gel-2 groups showed a significantly increasing trend compared to the positive control group (FIG. 5C).

Particularly, in the gel-2 group, an additional rise of IgG2a, known to demonstrate a protective effect against the onset of allergic inflammation via a mechanism similar to IgG1, was observed compared to the positive control group (FIG. 5D).

Based on these results, it was confirmed that the developed gel-1 and gel-2 groups can evoke an immune response with the necessary protective effects similar to those of conventional injection immunotherapies, while reducing unnecessary allergic immune reactions. Hence, they can be used as safer therapeutic agents that can replace the conventional immunotherapeutics.

### Example 6. Confirmation of Nasal Mucosal Symptom Relief Effect of HA Hydrogel-HDM Immunotherapeutic Agent

The effect of the HA hydrogel-HDM immunotherapeutic agent manufactured according to Example 1 on the nasal mucosa was confirmed. Specifically, after the immunotherapy, the nasal mucosa was washed and the number of eosinophils causing allergic inflammation present in the wash solution was compared using H&E staining. Also, the epithelial cells of the nasal mucosa were analyzed by tissue staining, where eosinophil infiltration was stained with H&E, mast cells with toluidine blue, and goblet cells with PAS.

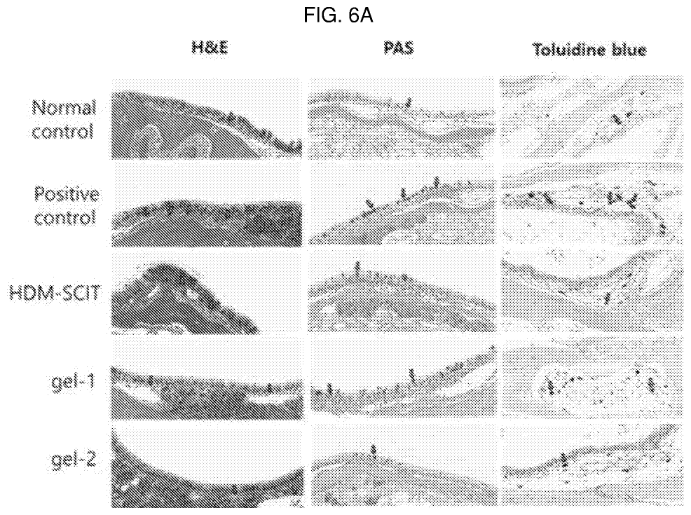

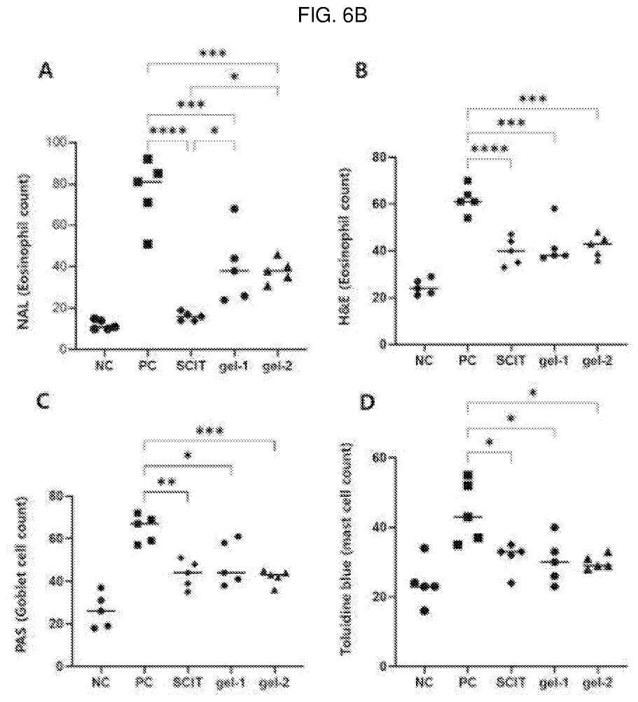

As a result, after immunotherapy of the gel-1 and gel-2 groups, the number of eosinophils in the nasal mucosal wash solution significantly decreased compared to the positive control group (FIG. 6A and FIG. 6B-A).

In tissue staining observations of the nasal mucosal epithelial cells, eosinophil infiltration causing allergic inflammation (H&E staining) significantly decreased in both the gel-1 and gel-2 groups compared to the positive control group, proving the therapeutic effect (FIG. 6A and FIG. 6B-B).

Furthermore, both the gel-1 and gel-2 groups showed a significant decrease in the number of goblet cells (goblet cell count, PAS staining) causing persistent rhinorrhea and nasal congestion in allergic rhinitis patients compared to the positive control group, confirming the effect of reducing mucus secretion by immunotherapy (FIG. 6A and FIG. 6B-C).

Lastly, the number of mast cells (mast cell count, toluidine blue staining) secreting inflammatory substances such as histamine and causing persistent pruritus and inflammation also significantly decreased in both the gel-1 and gel-2 groups compared to the positive control group, confirming the effect of relieving allergic inflammatory responses (FIG. 6A and FIG. 6B-D).

These results proved that both the gel-1 and gel-2 groups have a therapeutic effect of relieving the symptoms of allergic rhinitis, and they showed the same effect as SCIT group, which was known as the only fundamental treatment method for allergies.

### Example 7. Verification of the Cytokine Modulation Effect of the HA Hydrogel-HDM Immunotherapeutic Agent

The cytokine modulation effect of the HA hydrogel-HDM immunotherapeutic agent, prepared according to Example 1, was verified. Specifically, after culturing the spleen cells of the mouse in Example 2, splenocytes were stimulated with house dust mite antigens, and cytokine analysis was conducted.

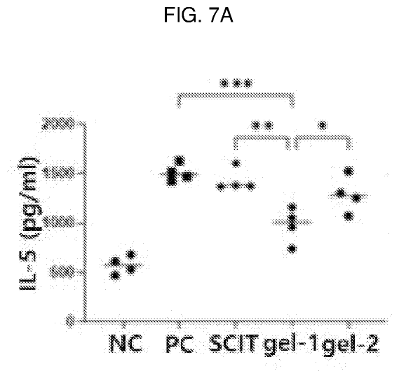

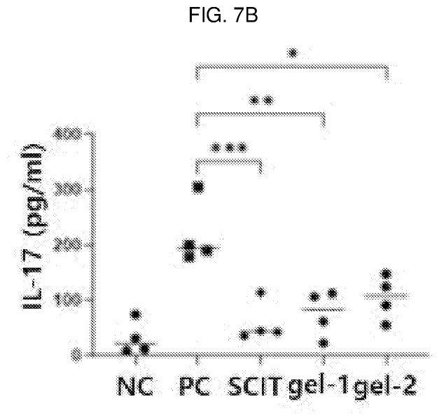

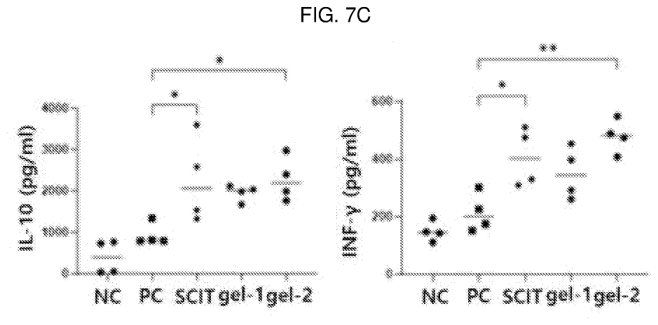

As a result, for the typical type 2 cytokine related to allergic responses, IL-5, both the gel-1 and gel-2 groups showed significant reduction effects compared to the positive control group. Especially, the gel-1 group showed statistically significant IL-5 reduction effect not only compared to the positive control group but also compared to the SCIT group (FIG. 7A). In addition, for another cytokine related to allergic responses, IL-17, a significant reduction was confirmed in all immunotherapy groups (FIG. 7B). Lastly, for IL-10 and IFN-v, known to increase due to the induction effect of immune tolerance after immunotherapy, both the gel-1 and gel-2 groups showed increasing trends compared to the positive control group. In particular, the gel-2 group was confirmed to show statistically significant increases in both IL-10 and INF-v compared to the positive control group (FIG. 7C).

In conclusion, the HA hydrogel-HDM according to the present invention, despite the inability to maintain its form for a long time due to the property of hyaluronic acid being easily degraded and absorbed into surrounding tissues in the human body, can induce immune tolerance through consistent degradation over a long period, by using the crosslinking method by riboflavin, a photoinitiator, and visible light, which can be easily injected through a syringe and solidified in the subcutaneous layer without any foreign body reactions. Therefore, it has been confirmed that this composition can continuously maintain its form in the body while maintaining the biocompatibility of the material, and can gradually deliver the drug. Furthermore, even when the injectable HA hydrogel and house dust mite crude extracts are mixed, low viscosity is maintained allowing injection through a syringe, and it was confirmed that foreign body reactions, inflammatory responses, and allergic hypersensitivity do not occur due to the solution injection. Thus, it is expected to be utilized as an immunotherapeutic agent that can overcome the disadvantages of conventional subcutaneous immunotherapy, such as dangerous side effects like anaphylaxis and the need for multiple repeat injections.

The description of the present invention as stated above is for illustrative purposes only, and a person having ordinary skill in the art to which the present invention pertains will understand that the technical idea or essential characteristics of the present invention can be easily modified into other specific forms without changing them. Therefore, the examples described above should be understood as illustrative in all aspects and not limited.

What is claimed is:

1. A method for treating immunotherapy of allergic diseases, the method comprising administering a composition comprising, crosslinked product of house dust mite antigens and hyaluronic acid hydrogels as an active ingredient to a subject in need thereof.

2. The method of claim 1, wherein the hyaluronic acid is substituted with a thiol group or a methacrylate group.

3. The method of claim 1, wherein the hydrogel comprises hyaluronic acid substituted with a thiol group (SH-HA) and hyaluronic acid substituted with a methacrylate group (MA-HA).

4. The method of claim 3, the volume ratio of hyaluronic acid substituted with a thiol group and hyaluronic acid substituted with a methacrylate group is 1:1.

5. The method of claim 1, wherein the hydrogel comprises hyaluronic acid and polyethylene glycol.

6. The method of claim 5, wherein the hyaluronic acid is substituted with a methacrylate group.

7. The method of claim 5, wherein polyethylene glycol is substituted with a thiol group.

8. The method of claim 5, the volume ratio of hyaluronic acid and polyethylene glycol is 1:1 to 4:1.

9. The method of claim 1, wherein the 750 μg of the house dust mite antigen is comprised based on 100 μL of the hyaluronic acid.

10. The method of claim 1, wherein the antigen is a crude extract.

11. The method of claim 1, wherein the composition further comprises a photoinitiator.

12. The method of claim 11, wherein the photoinitiator is riboflavin.

13. The method of claim 1, wherein the crosslinking of the crosslinked product is induced by visible light.

14. The method of claim 1, wherein the house dust mite is any one or more selected from the group consisting of Dermatophagoides pteronyssinus, Dermatophagoides farinae, Dermatophagoides microceras, and Euroglyphus maynei.

15. The method of claim 1, wherein the composition further comprises an adjuvant.

16. The method of claim 1, wherein the allergic disease is induced by house dust mites.

17. The method of claim 1, wherein the allergic disease is any one or more selected from the group consisting of rhinitis, asthma, conjunctivitis, urticaria, contact dermatitis, and atopic dermatitis.

18. An immunotherapeutic kit for treating allergic diseases comprising a crosslinked product of house dust mite antigens and hyaluronic acid hydrogels, and instructions.

19. The kit of claim 18, wherein the antigen is a crude extract.

20. A composition for immunotherapy of allergic diseases, which comprises a crosslinked product of house dust mite antigens and hyaluronic acid hydrogels as active ingredients.
